(19) United States
(12) Patent Application Publication
(10) Pub. No.: US 2025/0074760 A1
(43) Pub. Date: Mar. 6, 2025

(54) BEVERAGE DISPENSING SYSTEM WITH PERSONALIZED HYDRATION RECOMMENDATIONS

(71) Applicant: THE COCA-COLA COMPANY, Atlanta, GA (US)

(72) Inventors: HyunGyu SUH, Atlanta, GA (US); Haiyu REN, Marietta, GA (US); Matthew Charles KOCHEM, Jersey City, NJ (US); Yu SHI, Marietta, GA (US)

(21) Appl. No.: 18/726,287

(22) PCT Filed: Jan. 3, 2023
(86) PCT No.: PCT/US2023/060034

§ 371 Date: Jul. 2, 2024

Related U.S. Application Data
(60) Provisional application No. 63/296,236, filed on Jan. 4, 2022.

(51) Int. Cl. B67D 1/08; A61B 5/00; A61B 5/11; A61B 5/145; B67D 1/00; G05B 13/04

(52) U.S. Cl. CPC B67D 1/0888; A61B 5/1118; A61B 5/1123; A61B 5/14517; A61B 5/4875; B67D 1/0022; G05B 13/048

(57) ABSTRACT

A method for dispensing a personalized beverage mixture includes receiving a predicted volume of fluid lost by a user through sweat based on activity data indicating a type of activity performed and one or more parameters for the activity, receiving a measurement of an amount of electrolytes lost by the user through sweat, generating a recipe for the personalized beverage mixture based on the predicted volume of fluid and the measured amount of electrolytes lost by the user, the recipe including a second volume of fluid to replenish at least a portion of the predicted volume of fluid lost by the user and an amount of electrolyte additive to replenish at least a portion of the measured amount of electrolytes lost by the user, and dispensing the personalized beverage mixture according to the recipe.

### CROSS-REFERENCE TO RELATED APPLICATIONS

This application claims priority to and incorporates by reference U.S. Provisional patent application Ser. No. 63/296,236 filed on Jan. 4, 2022, and entitled BEVERAGE DISPENSING SYSTEM WITH PERSONALIZED HYDRATION RECOMMENDATIONS.

### BACKGROUND

The present disclosure relates generally to beverage dispensing systems and more specifically to a system and methods for generating and dispensing a personalized drink mixture. A beverage dispensing system may be configured to produce an additive-enhanced (e.g., flavored) beverage by mixing streams of additives (e.g., syrup, concentrate, sweetener, flavors, etc.) with water and/or other types of diluents (e.g., milk, coffee, etc.). For example, the beverage dispensing system may dispense separate streams of an additive and water which mix as the respective streams enter a container, such as a cup or bottle. In more recent years, certain beverage dispensing systems have been developed which allow a user to customize a beverage mixture to produce a beverage having, for example, a customized flavor profile. However, even beverage dispensing systems that allow users to customize their beverage may only allow for a limited number of additive combinations. Certain users (e.g., athletes) may benefit from additional customization options, such as the ability to include additives other than flavor.

Sports drinks are one example of a beverage that may benefit from more advanced customization options. For example, sports drinks are often intended to replace a signification portion of the fluid and electrolytes that an athlete loses through sweat. Heavy sweat loss without appropriate electrolyte replacement can lead to, for example, low blood sodium (i.e., hyponatremia), which can impair performance and health. Though rare, the risk for hyponatremia increases in athletes that produce a greater than average amount of sweat and/or those who consume large volumes of low electrolyte beverages (e.g., plain water). Accordingly, in some instances, an athlete's biometric data (e.g., weight, hydration level, heart rate, water intake, carbohydrate intake, protein intake, supplement intake, sodium intake, blood pressure, expended and intended exertion, temperature, blood oxygen levels, etc.) and nutritional intake may be closely monitored to tailor an individual hydration program to the athlete. However, generating individualized hydration programs is often time consuming and expensive, particularly for non-professional athletes that may not have access to trainers and coaches with extensive knowledge of sports nutrition.

### SUMMARY

One implementation of the present disclosure is a method for dispensing a personalized beverage mixture. The method can include receiving a predicted volume of fluid lost by a user through sweat based on activity data indicating a type of activity performed and one or more parameters for the activity, receiving a measurement of an amount of electrolytes lost by the user through sweat, generating a recipe for the personalized beverage mixture based on the predicted volume of fluid and the measured amount of electrolytes lost by the user, the recipe including a second volume of fluid to replenish at least a portion of the predicted volume of fluid lost by the user and an amount of electrolyte additive to replenish at least a portion of the measured amount of electrolytes lost by the user, and dispensing the personalized beverage mixture according to the recipe.

In some embodiments, the one or more activity parameters include at least one of a duration of the activity, an activity intensity, or ambient environmental conditions.

In some embodiments, the activity data is provided as a user input to a user device operated by the user or is automatically collected by the user device as the user performs the activity.

In some embodiments, the method further includes receiving physical parameters for the user including at least one of a height, a weight, or a gender of the user. In some such embodiments, the amount of electrolytes lost by the user is determined based further on the at least one of the height, the weight, or the gender of the user

In some embodiments, the measurement of the amount of electrolytes lost by the user through is collected by a sensor positioned on the skin of the user.

In some embodiments, the sensor is a chemical electrolyte sensor configured to display a visual indicator of the amount of electrolytes lost.

In some embodiments, the sensor an electronic electrolyte sensor configured to wirelessly transmit data.

In some embodiments, the method further includes displaying, via a user interface, an indication of the volume of fluid for the personalized beverage mixture.

In some embodiments, the method further includes displaying, via a user interface, an indication of a dehydration risk for the user.

In some embodiments, the method further includes receiving a user input indicating at least one of a flavor or a sweetener to add to the fluid. In some such embodiments, the recipe for the personalized beverage mixture further includes an amount of the flavor or the sweetener.

Another implementation of the present disclosure is a method for dispensing a personalized beverage mixture. The method can include receiving a predicted volume of fluid and a predicted amount of electrolytes lost by a user through sweat based on activity data indicating a type of activity performed and one or more parameters for the activity, generating a recipe for the personalized beverage mixture based on the predicted volume of fluid and the predicted amount of electrolytes lost by the user, the recipe including a second volume of fluid to replenish at least a portion of the predicted volume of fluid lost by the user and an amount of electrolyte additive to replenish at least a portion of the predicted amount of electrolytes lost by the user, and dispensing the personalized beverage mixture according to the recipe.

In some embodiments, the one or more activity parameters include at least one of a duration of the activity, an activity intensity, or ambient environmental conditions.

In some embodiments, the activity data is provided as a user input to a user device operated by the user or is automatically collected by the user device as the user performs the activity.

In some embodiments, the activity data further includes a measurement of electrolyte loss for the user based on data received from a wearable device worn by the user.

In some embodiments, the wearable device is a sweat sensor positioned on the skin of the user.

In some embodiments, a face of the wearable device is scanned using a camera of a user device operated by the user to determine the measurement of electrolyte loss.

In some embodiments, the method further includes displaying, via a user interface, an indication of the volume of fluid for the personalized beverage mixture.

In some embodiments, the method further includes displaying, via a user interface, an indication of a dehydration risk for the user.

In some embodiments, the method further includes receiving physical parameters for the user including at least one of a height, a weight, or a gender of the user. In some such embodiments, the amount of electrolytes lost by the user is determined based further on the at least one of the height, the weight, or the gender of the user.

Yet another implementation of the present disclosure is computer readable media having instructions stored thereon that, when executed by one or more processors, cause the one or more processors to perform operations including receiving activity data indicating a type of activity performed by a user and one or more parameters for the activity, predicting a first volume of fluid lost by the user through sweat during the activity, receiving, from a sensor positioned on the skin of the user, a measurement of an amount of electrolytes lost by the user through sweat, generating a recipe for a personalized beverage mixture based on the first volume of fluid and the measured amount of electrolytes lost by the user, the recipe including a second volume of fluid to replenish at least a portion of the first volume of fluid lost by the user and an amount of electrolyte additive to replenish at least a portion of the measured amount of electrolytes lost by the user, and transmitting a control signal to a drink dispensing device to cause the drink dispensing device to dispense the personalized beverage mixture according to the recipe.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is a block diagram of a system for generating and dispensing a personalized beverage mixture, according to some embodiments.

FIG. 2 illustrates an alternative configuration of the system of FIG. 1, according to some embodiments.

FIG. 3 is a block diagram of a hydration recommendation subsystem for generating a personalized beverage recipe, according to some embodiments.

FIG. 4 is a flow diagram of a process for generating and dispensing a personalized beverage mixture, according to some embodiments.

FIG. 5 is an example interface for providing activity data to generate a personalized beverage recipe, according to some embodiments.

FIG. 6 illustrates another example interface for providing activity data to generate a personalized beverage recipe, according to some embodiments.

FIGS. 7A and 7B illustrate yet another example interface for providing activity data to generate a personalized beverage recipe, according to some embodiments.

FIG. 8 is an example of a sensor for detecting a level of electrolytes lost by a user, according to some embodiments.

### DETAILED DESCRIPTION

Referring generally to the FIGURES, a system and methods for generating and dispensing a personalized beverage mixture are shown, according to some embodiments. In particular, the system and methods described herein may generate a personalized beverage recipe based on one or more variables relating to a user (e.g., physical and/or physiological characteristics) and/or an activity (e.g., exercise) performed by the user. Physical characteristics of the user may include, for example, the user's height, weight, etc. Activity parameters may include a type of activity, an amount of time spent performing the activity, an intensity of the activity, and more, as discussed in detail below. Based on the physical characteristics of the user and/or the activity parameters, an amount of fluid and/or an amount of electrolytes lost by the user through sweat can be predicted and/or determined, which can in turn be used to generate the personalized beverage recipe.

In contrast, many off-the-shelf or commercially available sports drinks aimed at replenishing an athlete's fluid and electrolyte levels do not account for the differences in fluid and electrolyte loss experienced by different athletes. Additionally, generic sports drinks often to not account for variations in activity type and intensity, or even environmental factors (e.g., wind, temperature, humidity, etc.) that may affect the amount of fluid and electrolytes an athlete loses. As an example, many generic sports drinks contain roughly 18 millimoles per liter (mmol/L) of sodium; however, the sodium concentration in human sweat has been known to vary from about 10 mmol/L to 90 mmol/L depending on the various physical characteristics and activity parameters identified above.

Even custom hydration programs created by trainers, coaches, etc., can face certain drawbacks. As mentioned above, it is often prohibitively expensive for non-professional athletes, and indeed for many professional athletes, to utilize a trained professional create a custom hydration program. Many custom hydration programs also require a user to order specific electrolyte additive mixtures, which can be costly and difficult to procure. The user must then find a source of clean water to mix their own custom beverage using the specific electrolyte additive. Additionally, certain hydration programs account only for the total volume of fluid lost due to sweat, and therefore do not provide an accurate and customized electrolyte balance for individual users.

To address these issues, the system and methods described herein can generate a personalized beverage recipe that provides a specific and tailored volume of fluid and electrolyte additive to aid the user in recovery. As described in detail below, the personalized recipe can account for variations in the physical attributes of various users and can also account for variations in activity intensity, duration, environmental factors, etc., as well as individual preferences such as flavor, carbohydrate level, etc. The personalized beverage recipe can then be utilized to automatically dispense a personalized beverage mixture having a specific volume of fluid and electrolytes, among other additives, for the user. Additional features and advantages of the dynamic drinking system will be discussed in greater detail below.

Personalized Beverage Dispensing System

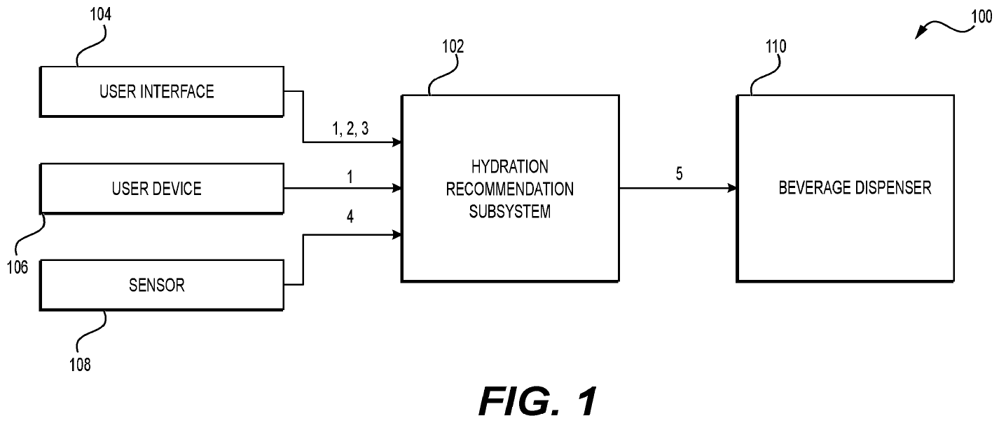

Referring first to FIG. 1, a block diagram of a system 100 for generating and dispensing a personalized beverage mixture is shown, according to some embodiments. In particular, system 100 may be configured to first generate a recipe for a personalized beverage mixture based on various parameters for an activity performed by a user and, in some cases, based on physical characteristics of the user. The recipe may define a personalized combination (e.g., ratio and amount) of fluid (e.g., water) and electrolyte additive, along with various other additives such as flavor, sugar, etc., that is tailored to the needs of the user. Subsequently, system 100 may automatically dispense the personalized beverage mixture based on the recipe. Users, as described herein, may be athletes or any other persons performing an activity (e.g., activity) or task over a period of time. However, it will be appreciated that any person, even one that is not performing or that has not performed an activity, may use and benefit from the advantages of system 100 described herein.

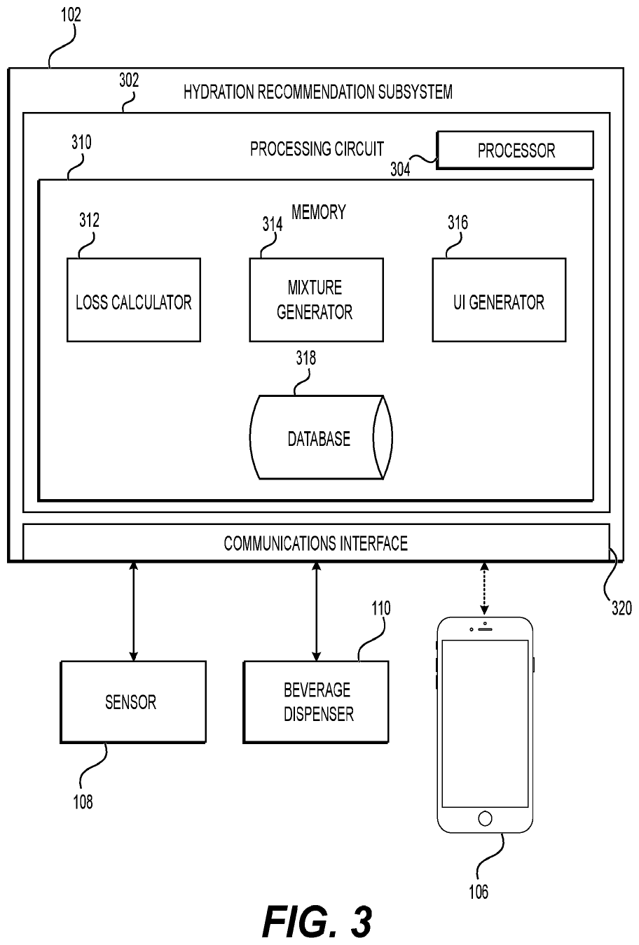

System 100 is shown to include a hydration recommendation subsystem 102 configured to generate a recipe for the personalized beverage mixture. Subsystem 102, described in greater detail below with respect to FIG. 3, may receive activity data and/or user data from one or more of a user interface 104, a user device 106, or a sensor 108, which is then used to generate the recipe. In FIG. 1, for example, activity data (1) and user data (2) are shown to be received by subsystem 100 from one or both of user interface 104 and wearable device 106. In some embodiments, additional preferences (3) of the user, such as flavorings, sweeteners, or other additives to be included in the personalized beverage mixture, may also be received from user interface 104.

As described herein, activity data may be any data relating to any activity performed by a user (e.g., an athlete), such as a type of activity, a duration of the activity, an activity intensity, ambient environmental conditions while the activity was performed (e.g., wind speed, ambient air temperature, humidity, terrain, etc.), etc. In some embodiments, activity data is specific to the type of activity (e.g., exercise) performed. Activity data for a user that is running on a treadmill, for example, may include a speed and/or incline of the treadmill. In some embodiments, at least a portion of the activity data is recorded by user device 106, as described below. In some embodiments, at least a portion of the activity data is entered by a user into user interface 104, also described below. However, it will be appreciated that user interface 104 and user device 106 may be utilized cooperatively to collect various activity data.

User data may be any data relating to physical or physiological characteristics of the user. In some embodiments, user data includes physical characteristics such as a height and weight of the user but can also include an age and/or gender of the user. In this regard, the user data may include any physical characteristics which impact the amount of fluid and/or electrolytes lost by the user during the period activity. For example, a large male athlete may lose a greater volume of fluid through sweat than, for example, a smaller female athlete. In some embodiments, user data includes physiological characteristics of the user such as resting heart rate, maximum heart rate, typical blood oxygen levels, etc. Electrolytes, as described herein, may include one or more of sodium, calcium, potassium, chloride, phosphate, and magnesium.

User interface 104 may be any device capable of receiving user inputs (e.g., including activity and/or user data) and, in some embodiments, displaying data and graphics. Accordingly, user interface 104 may include a user input device (e.g., a keypad, a keyboard, a mouse, a joystick, buttons, switches, a touchscreen, etc.) and a display (e.g., any type of LED or LCD screen). User interface 104 may also include a processor (e.g., a general-purpose processor, an application specific integrated circuit (ASIC), one or more field programmable gate arrays (FPGAs), a group of processing components, or other suitable electronic processing components) and memory (e.g., RAM, ROM, Flash memory, hard disk storage, etc.) capable of interpreting received user input and/or generating and displaying graphical interfaces. In the example of FIG. 1, user interface 104 is shown as a separate component from subsystem 102. For example, user interface 104 may be a mobile phone, an electronic tablet, a laptop, a desktop computer, a workstation, or the like that is communicably coupled to subsystem 102. However, in other embodiments, user interface 104 is a component of subsystem 102 itself. For example, user interface 104 may be coupled directly to, or mounted on, subsystem 102.

User device 106 may be any computing device capable of being carried by the user and, in general, may be configured to receive and/or record activity and user data. Accordingly, user device 106 can include a processor (e.g., a general-purpose processor, an application specific integrated circuit (ASIC), one or more field programmable gate arrays (FPGAs), a group of processing components, or other suitable electronic processing components) and memory (e.g., RAM, ROM, Flash memory, hard disk storage, etc.) for storing and executing instructions (e.g., programs or software applications). In some embodiments, user device 106 also includes a user interface (e.g., similar to or the same as user interface 104), such as a touchscreen, allowing the user to view information and/or provide inputs. For example, user device 106 may be a smartphone carried by the user (e.g., in a pocket) or a smartwatch worn by the user.

User device 106 may be configured to record activity data either responsive to a prompt from a user or automatically. Accordingly, user device 106 may include various sensors, transceivers, or other components for recording activity data. For example, user device 106 may include a GPS transceiver and/or accelerometer for recording the user's movements (e.g., pace, number of steps, distance traveled, etc.). In another example, user device 106 may include various sensors for measuring the user's heart rate, blood oxygen, or other physiological parameters (e.g., optical SpO2 sensors, optical pulse sensors, etc.). In some embodiments, user device 106 can automatically detect when a user has started an activity or workout based on various sensor data. For example, user device 106 may determine that the user is exercising if the user's heart rate increases, often in conjunction with an increase in movement.

In some embodiments, user device 106 is configured to receive a user input identifying a type of activity (e.g., running, rowing, biking, etc.). In other embodiments, user device 106 can automatically determine a type of activity based on movement and/or location data. For example, a speed of the user may indicate whether the user is running or biking. In another example, location data may indicate whether the user is on or is following a roadway, sidewalk, trail, etc. In some embodiments, movement data and heart rate for the user can also be used to determine an intensity of the activity. When activity is detected, user device 106 may also record an amount of time (i.e., duration) that the user spends performing the activity. Additionally, user device 106 may be communicably coupled to an external network, such as the Internet, from which environmental data may be retrieved. For example, user device 106 may receive weather data from an online weather service indicating temperature, humidity, wind speed, etc.

As briefly mentioned above, user interface 104 and user device 106 are shown in the example of FIG. 1 to be separate components; however, it will be appreciated that user interface 104 and user device 106 can, in some embodiments, be parts of a common device. For example, both user interface 104 and user device 106 may be components of a user device, such as a smartphone or smartwatch carried by the user. In another example, as mentioned above, user interface 104 may be a component of subsystem 102, while user device 106 is a separate device carried by the user. Accordingly, all such possible configurations of system 100 are contemplated herein.

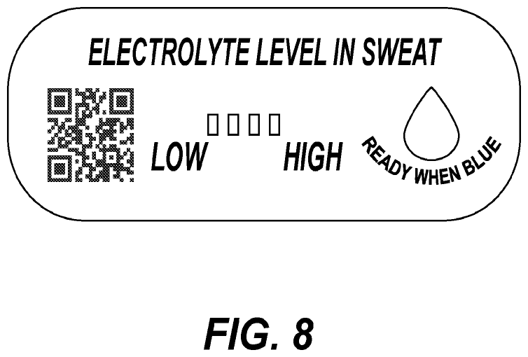

Sensor 108 may be a sweat sensor or similar device configured to measure an amount of electrolytes lost by the user (e.g., through sweat). In particular, sensor 108 may be worn on the skin of the user and may detect the amount of electrolytes expelled through the user's skin. In some embodiments, sensor 108 is a chemical electrolyte sensor as described in one or both of U.S. Provisional Patent Application No. 63/138,964, filed Jan. 19, 2021, and U.S. Provisional Patent Application No. 63/276,860, filed Nov. 8, 2021, both of which are incorporated by reference herein in their entireties. In some such embodiments, sensor 108 may react with the electrolytes present in a sample of the user's sweat to determine a level of one or more electrolytes in the sweat. The electrolyte level may be displayed, for example, on a surface of sensor 108, as shown in FIG. 8, described in greater detail below. Accordingly, in some such embodiments, an electrolyte measurement (4) may be received by subsystem 102 via a user input (e.g., to either of user interface 104 or user device 106) of the electrolyte level or by scanning the face of sensor 108, such as with a camera of user device 106.

In some embodiments, sensor 108 is an electrical electrolyte sensor as described in PCT Patent Application No. PCT/US21/55417, filed Oct. 18, 2021, which is also incorporated by reference herein in its entirety. For example, sensor 108 may measure various characteristics of the user's sweat, such as one or more of impedance, conductivity, refraction, temperature, or any combination thereof, to determine the amount of electrolytes lost by the user. In some embodiments, sensor 108 can wirelessly transmit an indication of the amount of electrolytes, either directly to system 102 (e.g., as electrolyte measurement (4)) or indirectly through user device 106 (not shown).

Upon receiving activity data (1), user data (2), additional preferences (3), and/or electrolyte measurement (4), subsystem 102 may be configured to generate a unique recipe for a personalized drink mixture. As discussed briefly above, the recipe may indicate at least a volume of fluid for the personalized drink mixture and an amount of electrolyte additive to mix with the volume of fluid. The volume of fluid for the personalized drink mixture, for example, may be intended to completely, or at least partially, replenish the volume of fluid lost by the user through sweat. For example, if it is determined (e.g., using activity data (1) and/or user data (2)) that the user has lost or is predicted to lose 500 mL of fluid through sweat, then the volume of fluid for the personalized drink mixture may be at or around 500 mL; although, in some cases, it may be beneficial for the volume of fluid for the personalized drink mixture to be even greater or less than the volume of fluid lost through sweat.

In some embodiments, the amount of electrolyte additive to mix with the volume of fluid is determined based, at least in part, on the total volume of fluid for the personalized drink mixture. For example, the amount of electrolyte additive may be determined based upon a desired concentration of electrolytes for the personalized drink mixture. As mentioned above, many commercially available sports drinks include an 18 mmol/L concentration of electrolytes; however, subsystem 102 may determine that a specific user requires a 25 mmol/L concentration of electrolytes. Accordingly, in this example, subsystem 102 may determine the amount of electrolyte additive to mix with the volume of fluid for the personalized drink mixture to achieve the 25 mmol/L concentration.

In other embodiments, the amount of electrolyte additive is determined without consideration of the final volume of fluid in the personalized drink mixture. For example, subsystem 102 may directly determine the amount of electrolytes lost by the user such that the corresponding amount of electrolyte additive can be mixed with any amount of fluid. In this manner, the final concentration of electrolytes in the personalized drink mixture may [not?] be based on the total volume of fluid for the personalized drink mixture.

In either case, the total amount of electrolyte additive may be less than an upper threshold to avoid generating a recipe that would be too concentrated for consumption. In some embodiments, the upper threshold is about 60 mmol/L of electrolyte additive. As used herein, “about” means ±20% of an associated value, unless otherwise specified. For example, about 60 mmol/L encompasses the range between 48 mmol/L to 72 mmol/L. Concentrations beyond this upper limit may, for example, may be too salty for many users or otherwise adversely affect the flavor profile of the dispensed beverage.

Once generated, the recipe (5) may be transmitted to a beverage dispenser 110, which is configured to dispense one or more fluids and additives according to the recipe. Specifically, beverage dispenser 110 may dispense a base fluid, such as water, and one or more additives (e.g., flavor, nutrients, sweeteners, etc.) including the electrolyte additive to create the personalized drink mixture. In some embodiments, beverage dispenser 110 is functionally equivalent to, or the same as, the beverage dispensers described in detail in one or both of U.S. patent application Ser. No. 16/639,361, filed Feb. 20, 2020, and U.S. patent application Ser. No. 17/257,958, filed Jan. 5, 2021, both of which are incorporated by reference herein in their entireties.

While shown as separate components in the example of FIG. 1, it should be appreciated that one or both of subsystem 102 and user interface 104 may be components of beverage dispenser 110. In other words, one or both of subsystem 102 and user interface 104 may be integrated into beverage dispenser 110. As an example, user interface 104 may be mounted directly onto beverage dispenser 110 (e.g., onto a front face of beverage dispenser 110) such that a user can input activity and/or user data directly to beverage dispenser 110. In another example, subsystem 102 may be integrated into beverage dispenser 110 such that one or more processors and/or memory devices of beverage dispenser 110 implement the features of subsystem 102 described above.

In yet another embodiment, subsystem 102 may be, or may be hosted on, a server or other computing device that is remote from beverage dispenser 110 (i.e., a remote server). In such embodiments, subsystem 102 may be in communication with any of the components of system 100 via any suitable wired or wireless network. For example, subsystem 102 may receive activity and/or user data (1, 2) from one or both of user interface 104 and user device 106 via an Internet connection and may transmit the recipe (5) to beverage dispenser 110 via the Internet or another, private connection. In some embodiments, subsystem 102 may transmit a predicted volume of fluid and/or measured or predicted electrolyte loss due to sweat to beverage dispenser 110, which is then used by the beverage dispenser 110 to determine the recipe (5). It will be appreciated that all such possible configurations of beverage dispenser 110, and thereby system 100, are contemplated herein.

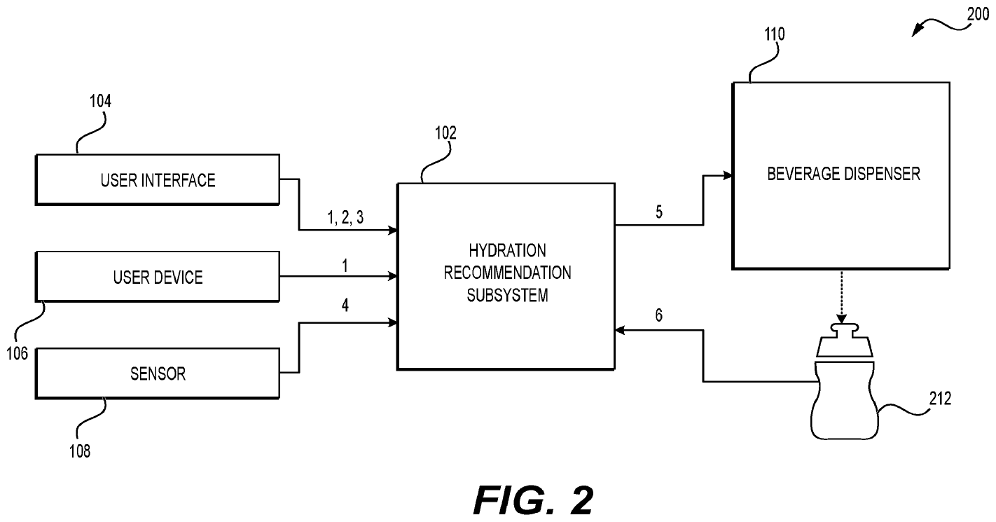

Referring now to FIG. 2, an alternative configuration 200 of system 100 is shown, according to some embodiments. In many respects, alternative configuration 200 is similar to the configuration of system 100 described above with respect to FIG. 1. For example, user interface 104, user device 106, and sensor 108 may provide activity data (1), user data (2), additional preferences (3), and/or electrolyte measurements (4) to subsystem 102, which generates a recipe (5) that is then provided to beverage dispenser 110. However, alternative configuration 200 is also shown to include a smart vessel 212 into which the personalized beverage mixture is dispensed.

For simplicity, smart vessel 212 will be described herein as a form of reusable water bottle having a resealable cap. However, it will be appreciated that smart vessel 212 may be any type of cup, mug, bottle, or other container capable of holding a fluid (e.g., for human consumption). In general, smart vessel 212 may include a processor, memory, and/or various sensors for determining a volume of fluid in smart vessel 212 and/or for detecting when, and thereby how often, the user is drinking. For example, a sensor positioned in a cap of smart vessel 212 may continuously or regularly (e.g., at a regular time interval) measure a level of fluid in smart vessel 212, which can be evaluated (e.g., by comparing the level at various time steps) to determine whether the user has consumed any of the fluid. To continue this example, a difference between the fluid level at a first and second time step may indicate that the user has taken a drink (e.g., if the fluid level is lower at the second time step) or that smart vessel 212 has been filled (e.g., if the fluid level is higher at the second time step).

In some embodiments, smart vessel 212 can transmit measurements of the fluid level (6) to an external device (e.g., a user device such as user device 106), such that the external device can perform various calculations using the fluid level measurements. In this manner, the external device can handle the bulk of the computations required to determine changes in the fluid level, thereby reducing the computational requirements, battery usage, etc., for smart vessel 212. As shown in FIG. 2, for example, smart vessel 212 may transmit fluid level measurements (6) to subsystem 102. Subsystem 102 can then utilize the fluid level measurements to determine not only the fill level of smart vessel 212 but also the rate at which the user is consuming the fluid.

In some embodiments, subsystem 102 is configured to remind the user to drink from smart vessel 212. For example, subsystem 102 may determine that the user is not on track to meet a daily fluid level intake, or to consume a recommended amount of fluid to replenish losses from activity, and may generate and display (e.g., via user interface 104 or by transmitting a command to user device 106) a notification prompting the user to drink. In some embodiments, subsystem 102 can also determine a portion of the fluid in smart vessel 212 that the user must consume to replenish loses from activity (e.g., based on the recipe generated by subsystem 102, as described above). For example, subsystem 102 may provide an indication to the user (e.g., via user interface 104 or by transmitting a command to user device 106) that they should consume a percentage of the fluid in smart vessel 212 to replenish the loses due to sweat.

In some embodiments, a user profile is maintained by subsystem 102 or by another remote computing device (not shown) that tracks the personalized beverage mixture dispensed by beverage dispenser 110. For example, each time a personalized beverage mixture is dispensed from beverage dispenser 110, a user profile may be updated to include an indication of the volume of fluid dispensed, the amount of electrolyte additive dispensed, and/or the recipe used to generate the personalized beverage mixture. In some embodiments, the user's consumption of the personalized beverage mixture is also tracked based on readings of the fluid level in smart vessel 212 to monitor and/or predict a level of hydration for the user. Additional details and features of user profiles are discussed in greater detail below.

Referring now to FIG. 3, a block diagram illustrating subsystem 102 in greater detail is shown, according to some embodiments. As described above, subsystem 102 may be configured to generate a unique recipe for a personalized drink mixture based upon one or more of activity data, user data, and additional preferences of the user (e.g., flavors, sweeteners, etc.). In some embodiments, the recipe is also generated based on an electrolyte measurement from a sweat sensor. In this way, the unique recipe can improve a user's recovery or rehydration (e.g., from a period of activity) by tailoring the recipe to the user's individual needs. Additionally, subsystem 102 may cause beverage dispenser 110 to automatically dispense a personalized beverage mixture based on the recipe, which may be significantly more convenient for the user over other hydration programs which require the user to mix their own beverage.

Subsystem 102 is shown to include a processing circuit 302 that further includes a processor 304 and a memory 310. Processor 304 can be a general-purpose processor, an application specific integrated circuit (ASIC), one or more field programmable gate arrays (FPGAs), a group of processing components, or other suitable electronic processing components. In some embodiments, processor 304 is configured to execute program code stored on memory 310 to cause subsystem 102 to perform one or more operations. Memory 310 can include one or more devices (e.g., memory units, memory devices, storage devices, etc.) for storing data and/or computer code for completing and/or facilitating the various processes described in the present disclosure.

In some embodiments, memory 310 includes tangible, computer-readable media that stores code or instructions executable by processor 304. Tangible, computer-readable media refers to any media that is capable of providing data that causes the subsystem 102 (i.e., a machine) to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 310 can include random access memory (RAM), read-only memory (ROM), hard drive storage, temporary storage, non-volatile memory, flash memory, optical memory, or any other suitable memory for storing software objects and/or computer instructions. Memory 310 can include database components, object code components, script components, or any other type of information structure for supporting the various activities and information structures described in the present disclosure. Memory 310 can be communicably connected to processor 304, such as via processing circuit 302, and can include computer code for executing (e.g., by processor 304) one or more processes described herein.

While shown as individual components, it will be appreciated that processor 304 and/or memory 310 can be implemented using a variety of different types and quantities of processors and memory. For example, processor 304 may represent a single processing device or multiple processing devices. Similarly, memory 310 may represent a single memory device or multiple memory devices. Additionally, in some embodiments, subsystem 102 may be implemented within a single computing device (e.g., one server, one housing, etc.). In other embodiments subsystem 102 may be distributed across multiple servers or computers (e.g., that can exist in distributed locations). For example, subsystem 102 may include multiple distributed computing devices (e.g., multiple processors and/or memory devices) in communication with each other that collaborate to perform operations. In some embodiments, subsystem 102 is a component of beverage dispenser 110 and thus may share processor 304 and/or memory 310 with beverage dispenser 110. For example, beverage dispenser 110 may include one or more processors and memory devices that implement the features of subsystem 102 described herein.

Memory 310 is shown to include a loss calculator 312 configured to determine or predict a volume of fluid and an amount of electrolytes lost by a user due to sweat. In particular, loss calculator 312 may predict a volume of fluid lost by a user due to sweat based on activity data and/or user data. As described above, activity data can include at least a type of activity (e.g., running, biking, rowing, hiking, kayaking, etc.), a duration of the activity, an activity intensity (e.g., light, moderate, heavy), and ambient environmental conditions (e.g., wind speed, ambient air temperature, humidity, terrain, etc.), along with any other data that may be relevant in determining or predicting an amount of energy, and thereby a volume of sweat, expended by the user through activity. In some embodiments, additional activity data that is specific to the type of activity performed can also be used to predict the volume of fluid lost through sweat. For example, activity data for rowing may include an average rowing pace (e.g., in meters per second).

Loss calculator 312 may also utilize user data to predict the volume of fluid lost through sweat. User data can include physical characteristics of the user, such as height, weight, gender, age, etc., as well as physiological characteristics, such as resting heart rate, maximal oxygen consumption (e.g., VO₂ max), etc. In some embodiments, historical activity data is also used (e.g., average pace, average distance traveled, etc.). As discussed above, all of these activity and user parameters may affect the amount of fluid expelled by the user through sweat. In some embodiments, loss calculator 312 can predict the volume of fluid loss using a model for the user. For example, the activity and/or user data discussed above may be used as inputs to the model to predict (e.g., as an output of the model) the volume of fluid lost. Said model may be, in some embodiments, an artificial intelligence model, such as a neural network model or the like. In other embodiments, another type of algorithm for predicting the volume of fluid lost through sweat may be utilized.

In some embodiments, loss calculator 312 can also predict the amount of electrolytes lost by a user due through sweat based on the activity and/or user data. For example, intense activity (e.g., determined based on duration, pace, etc.) may cause a user to lose more sweat than light activity. In another example, environmental factors such as temperature, humidity, and wind speed may greatly affect the volume of sweat, and thereby electrolytes, lost by the user. Like the prediction for the volume of fluid lost, electrolyte loss may also be predicted using a model of the user or another type of algorithm. However, in some embodiments, loss calculator 312 may receive a measurement of electrolyte loss from sensor 108 which, as described above, may be a chemical or electrical sweat sensor.

In some such embodiments, where sensor 108 is a digital or electrical sweat sensor, the measure of electrolyte loss may be received wirelessly from sensor 108. For example, sensor 108 may transmit electrolyte data through a short-range or low-power wireless connection (e.g., Bluetooth®). In another example, user device 106 may be configured to wirelessly scan sensor 108 (e.g., using RFID or a similar technology) to receive the electrolyte loss data. In some embodiments, where sensor 108 is a chemical sweat sensor, user device 106 can be configured to scan (e.g., using a camera) sensor 108 to determine a level of electrolytes in the user's sweat. In other such embodiments, the user may manually enter the level of electrolytes shown on sensor 108 via a user interface presented on user device 106. Additionally details of sensor 108 are described in greater detail below with respect to FIG. 8.

In some embodiments, the volume of fluid lost through sweat may be calculated based at least in part on the measured electrolyte loss for the user. For example, loss calculator 312 may be configured to predict the volume of fluid lost through sweat based on a level of electrolytes present in the user's sweat. In some such embodiments, user data and/or activity data may be used to inform the prediction. For example, the user data and/or activity data may be used to predict a volume of fluid lost at various different electrolyte concentrations in the user's sweat.

Memory 310 is also shown to include a mixture generator 314 configured to generate a recipe for the personalize beverage mixture based on the fluid and electrolyte loss data determined by loss calculator 312. The recipe, as mentioned above, may be configured to replenish (i.e., replace) at least a portion of the fluid and/or electrolytes lost by the user. For example, if loss calculator 312 predicts that the user has lost 750 mL of fluid through sweat, then the recipe generated by mixture generator 314 may include a corresponding volume of fluid (e.g., water). It will be appreciated that the volume of fluid for the personalize beverage mixture may not necessarily be directly equivalent to the volume of fluid lost by the user through sweat. For example, the personalize beverage mixture recipe may include a lesser or greater volume of fluid. Mixture generator 314 may also be configured to determine an amount of electrolyte additive to add to the volume of fluid to achieve a desired electrolyte concentration. For example, if the recipe includes 500 mL of water and requires an electrolyte concentration of 20 mmol/L, then mixture generator 314 may determine that the recipe should include 10 mmol of electrolyte additive. As mentioned above, however, mixture generator 314 may account for an upper threshold to the electrolyte concentration to avoid generating a recipe that would be too concentrated (e.g., too salty) for consumption. In one example, the upper threshold is about 60 mmol/L of electrolyte additive.

In some embodiments, mixture generator 314 can also generate the recipe based on additional user defined additives. In other words, mixture generator 314 may include one or more of a flavor, a sweetener, or other additives as selected by a user in the generated recipe. For example, in addition to any electrolyte additive, a user may wish to include a flavoring and/or a sweetener (e.g., sugar or other carbohydrates) to enhance the personalized beverage mixture. In some embodiments, additional additives are selected by the user via a user interface presented on user device 106. In any case, after generating the recipe for the personalized beverage mixture, mixture generator 314 may be configured to transmit the recipe to beverage dispenser 110. In some such embodiments, receiving the recipe can cause beverage dispenser 110 to dispense the personalized beverage mixture. In other embodiments, such as when subsystem 102 is part of beverage dispenser 110, beverage dispenser 110 may simply initiate dispensing of the personalized beverage mixture responsive to the generation of the recipe.

In some embodiments, a generated recipe and/or any of the activity and user data discussed above may be stored in a database 318. For example, database 318 may maintain a log of a user's activity data for improving predictions of fluid and/or electrolyte loss over time. In some embodiments, user data may be stored in a user profile on database 318 such that the user does not need to regularly enter characteristics such as height, weight, etc. In other words, user characteristics may used to generate a user profile that can be subsequently accessed by the user (e.g., by entering credentials into a web portal or software application) to generate new recipes or to modify existing recipes. In this manner, after initially generating the user profile, only new activity data may be needed to generate a recipe for a personalize beverage mixture.

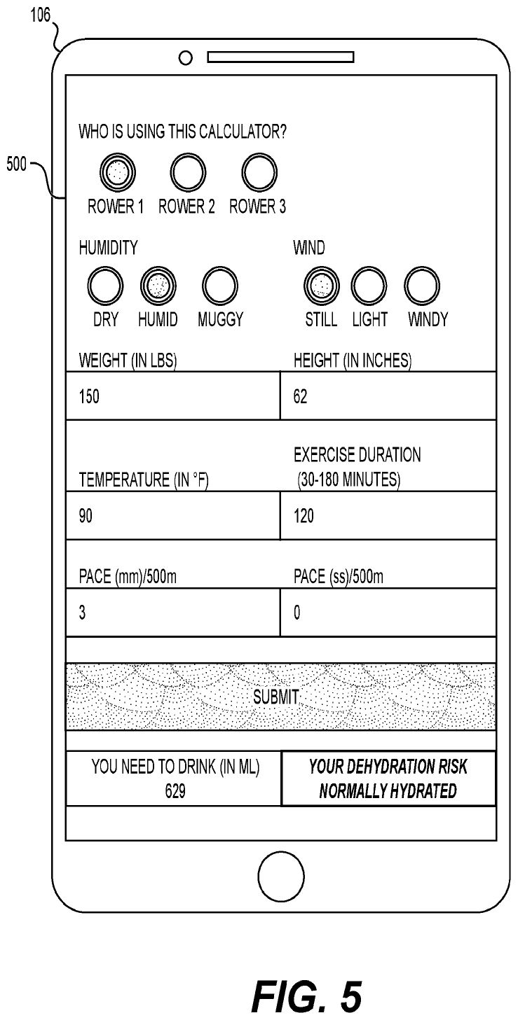

Memory 310 is also shown to include a user interface (UI) generator 316 configured to generate any of the user interfaces described herein. In particular, UI generator 316 may be configured to generate user interfaces that allow the user to input activity data, user data, and/or any additional preferences prior to generating a recipe for the personalized drink mixture. In some embodiments, UI generator 316 can also generate user interfaces that present the user with information relating to the recipe, such as a volume of fluid and/or an amount of electrolyte additive in the recipe. For example, the user may be presented with parameters for the recipe such that the user may manually mix the personalized drink mixture, if desired. In some embodiments, the user is presented with at least an indication of the volume of fluid that the user should consume to replace the predicted amount of fluid lost due to sweat, as described in greater detail below with respect to FIG. 5. In some embodiments, the user can also be alerted as to a dehydration risk.

Still referring to FIG. 3, subsystem 102 is also shown to include a communications interface 320 that can facilitate communications between subsystem 102 and any external components or devices. For example, communications interface 320 can provide means for transmitting data to, or receiving data from, sensor 108 and/or beverage dispenser 110, as described above. Accordingly, communications interface 320 can be or can include a wired or wireless communications interface (e.g., jacks, antennas, transmitters, receivers, transceivers, wire terminals, etc.) for conducting data communications. However, as mentioned above, it should be understood that subsystem 102 may be a component of beverage dispenser 110 and thus may communicate directly with beverage dispenser 110 (e.g., via a communication or data bus). For example, subsystem 102 may share processing components (e.g., processors and memory) and/or subsystem 102 may be implemented using the processor and memory of beverage dispenser 110.

In various embodiments, communications via communications interface 320 may be direct (e.g., local wired or wireless communications) or via a network (e.g., a WAN, the Internet, a cellular network, etc.). For example, communications interface 320 can include a WiFi transceiver for communicating via a wireless communications network. In another example, communications interface 320 may include cellular or mobile phone communications transceivers. In yet another example, communications interface 320 may include a low-power or short-range wireless transceiver (e.g., Bluetooth®). As described above, communications interface 320 may also include an internal data bus to couple subsystem 102 with various other processing devices and/or memory of beverage dispenser 110.

Personalized Beverage Recipe Generation

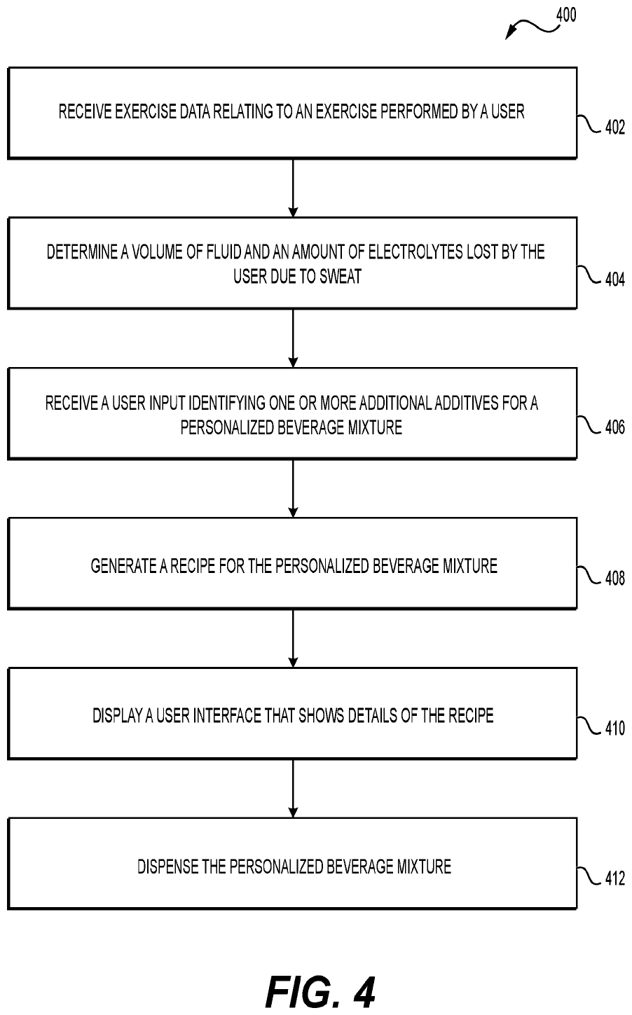

Referring now to FIG. 4, a flow diagram of a process 400 for generating and dispensing a personalized beverage mixture is shown, according to some embodiments. In some embodiments, process 400 is implemented by subsystem 102, as described above. For example, in instances where subsystem 102 is a component of beverage dispenser 110, process 400 may be directly or indirectly implemented by beverage dispenser 110. However, in various other embodiments, process 400 can be implemented by multiple components of system 100, as described above. For example, certain steps of process 400 may be performed by subsystem 102, while other steps may be performed by user device 106. It will also be appreciated that certain steps of process 400 may be optional and, in some embodiments, process 400 may be implemented using less than all of the steps.

At step 402, activity data relating to an activity performed by the user is received. In some embodiments, the activity data is received via a user input to a user device (e.g., user device 106), such as a smartphone and/or smartwatch. For example, the user device may display an interface (e.g., of a software application or a web interface) that includes multiple entry fields or other graphical elements that allow the user to identify activity data such as a type of activity, a duration of the activity, an activity intensity, ambient environmental conditions, pace, etc. In other embodiments, the activity data is received from the user device having been automatically or at least semi-automatically collected while the user performs the activity. For example, the user device (e.g., a smartwatch) may execute a software application that tracks the user's movements (e.g., speed, direction, distance, etc.) and/or physiological parameters (e.g., heart rate, blood oxygen, etc.). In some such embodiments, certain data may be automatically collected from a remote source. For example, the user device may retrieve environmental data from an online weather and/or mapping service. In still other embodiments, the activity data is received as a combination of user inputs and collected data. For example, certain activity data (e.g., duration, distance, pace, etc.) may be automatically collected by the user device while additional activity data (e.g., activity type and intensity) may be provided as a user input.

In some embodiments, step 402 also includes receive user data. As described above, user data may include any physical and/or physiological information about the user, such as height, weight, gender, and age. In some such embodiments, user data is provided as a user input to the user device. For example, the user data may be used to generate a user profile. The user profile may then be saved such that the user data may be retrieved at a later time period with requiring the user to provide additional inputs.

At step 404, a volume of fluid and a corresponding amount of electrolytes lost by the user through sweat is determined. As described above, the activity data and/or the user data may be used to predict (i.e., estimate) both the volume of fluid and the amount of electrolytes lost by the user through sweat while performing the activity. In some embodiments, the amount of electrolytes lost through sweat is measured by a sweat sensor positioned on a skin of the user. In some such embodiments, an indication of the amount of electrolytes lost is received wirelessly from the sweat sensor. In other such embodiments, the sweat sensor is scanned using a camera or other optical sensor of a user device to read an indication of the amount of electrolytes lost. For example, the sweat sensor may be a chemical sweat sensor that displays one or more color bars based on the level of electrolytes in a sample of sweat.

At step 406, a user input identifying one or more additional additives for the personalized beverage mixture is optionally received. In some embodiments, the user input is received from the user device in a similar manner to the activity and/or user data described above. The one or more additional additives may include, for example, flavorings, sweeteners, and the like that may enhance the personalized beverage mixture based on the user's taste. For example, it may be desirable to include a sugar or another calorie-dense sweetener (e.g., corn syrup) in the personalized beverage mixture to replenish carbohydrates burned by the user for energy while exercising.

At step 408, a recipe for the personalized beverage mixture is generated. In particular, the recipe may include a volume of fluid, an amount of electrolyte additive, and/or an amount of one or more additional additives to include in the personalized beverage mixture. The volume of fluid may correspond to an amount of fluid, such as water, to be consumed by the user to replenish at least a portion of the fluid lost through sweat. In some embodiments, the volume of fluid represents a total volume of the personalized beverage mixture intended to rehydrate the user. Likewise, the amount of electrolyte additive (e.g., sodium or a combination of electrolytes) may correspond to an amount of electrolytes lost by the user through sweat. In some embodiments, the recipe may indicate a specific amount of electrolyte additive (e.g., in mmol) to add to the volume of fluid. In other embodiments, the recipe may indicate a concentration of electrolyte in the final personalized beverage mixture. From the concentration of electrolyte, an amount of electrolyte additive may be determined.

At step 410, a user interface that shows details of the recipe is optionally displayed. The user interface, which may be displayed via a user device or on a display of a beverage dispenser, may indicate a volume of fluid that the user should consume to replenish the predicted volume of fluid lost through sweat. For example, the user interface may indicate that the user should “Drink Y mL of Fluid,” where Y is the volume indicated in the recipe. In some embodiments, the fluid may be a commercially available sports drink (e.g., assumed to have an electrolyte concentration of 18 mmol/L). Although, the fluid may more likely be the personalized beverage mixture. In some embodiments, the user interface can also indicate the concentration and/or total amount of electrolyte additive included in the personalized beverage mixture.

At step 412, the personalized beverage mixture is dispensed. In some embodiments, where process 400 is mainly implemented by a beverage dispenser, the beverage dispenser may dispense the personalized beverage mixture responsive to the generation of the recipe. In other embodiments, the recipe generated at step 408 may be transmitted to the beverage dispenser for later dispensing. In some such embodiments, the recipe may be stored (e.g., in database 318) such that the user may retrieve the recipe and cause beverage dispenser to dispense the personalized beverage mixture. For example, the user may need to travel to the beverage dispenser and, once arriving at the beverage dispenser, may interact with a user interface to cause the beverage dispenser to dispense the personalized beverage mixture. In other such embodiments, the beverage dispenser can dispense the personalized beverage mixture responsive to receiving the recipe.

Referring now to FIG. 5, an example interface 500 for providing activity data to generate a personalized beverage recipe is shown, according to some embodiments. In this example, interface 500 may be representative of a user interface displayed on user device 106, or any other portable user device (e.g., a smartphone or smartwatch). Interface 500 is shown to include multiple fields and/or graphical elements that allow the user to input information such as a user identification, environmental data (e.g., humidity, wind, and temperature), user data (e.g., weight and height), activity duration, and pace. In this specific example, interface 500 is shown to be populated with data relating to a first rower. Accordingly, it will be appreciated that certain fields of interface 500 may be different based on the specific user and/or the type of activity. For example, for a user that is cycling, interface 500 may include additional fields such as pace (e.g., in miles per hour), distance (e.g., in miles), elevation, etc.

Once fully populated, the user may select a submission button (e.g., “Submit”) to cause user device 106, or subsystem 102, to perform calculations using the entered activity and user data. In particular, the entered data may be used to predict a volume of fluid lost by the user due to activity and, in some cases, an amount of electrolytes lost by the user. Subsequently, a recipe for a personalized beverage mixture may be generated. In this example, the total volume of fluid included in the recipe is displayed in a field that indicates to the user that they should drink 629 mL of fluid (e.g., plain water, sports drink, or personalized beverage mixture) to replenish lost fluids. Interface 500 may also present an indication of dehydration risk for the user. In this case, the user is determined to be “normally hydrated,” or not at serious risk of dehydration.

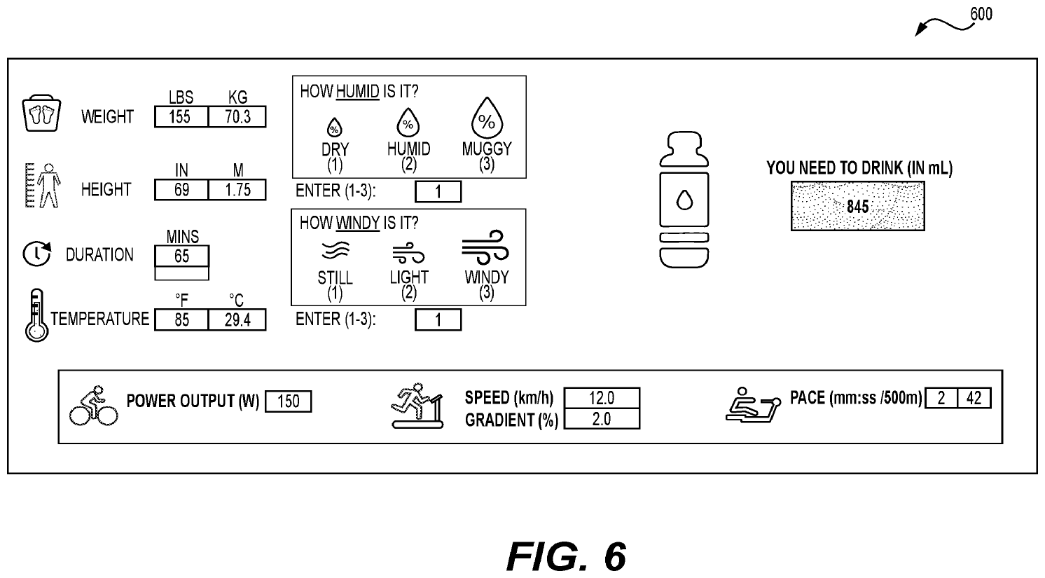

Referring now to FIG. 6, another example interface 600 for providing activity data to generate a personalized beverage recipe is shown, according to some embodiments. In particular, interface 600 may be an example of a web interface for receiving user inputs and displaying hydration data. In many respects, interface 600 may be similar to interface 500 described above, in that interface 600 includes multiple fields for user inputs of activity and user data. For example, interface 600 is shown to include fields for receiving a user's weight and height, as well as activity duration, ambient air temperature, humidity level, and wind level. Additionally, interface 600 is shown to include fields where the user can enter activity specific data, such as power output (e.g., for stationary cycling), speed and gradient (e.g., for running on a treadmill), and pace (e.g., for stationary rowing). In this example, based on the activity and user data, interface 600 provides an indication to the user that should drink 845 mL of fluid.

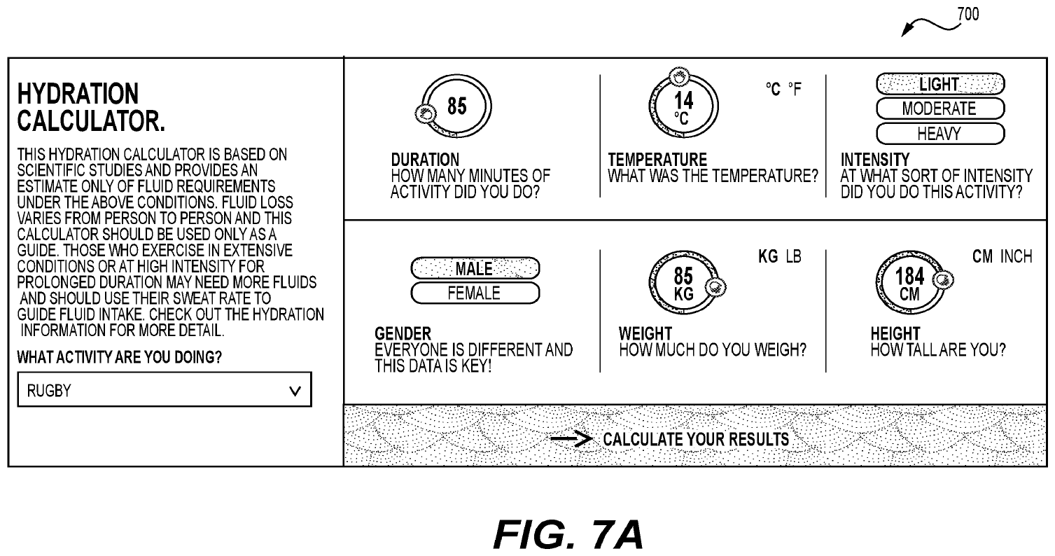

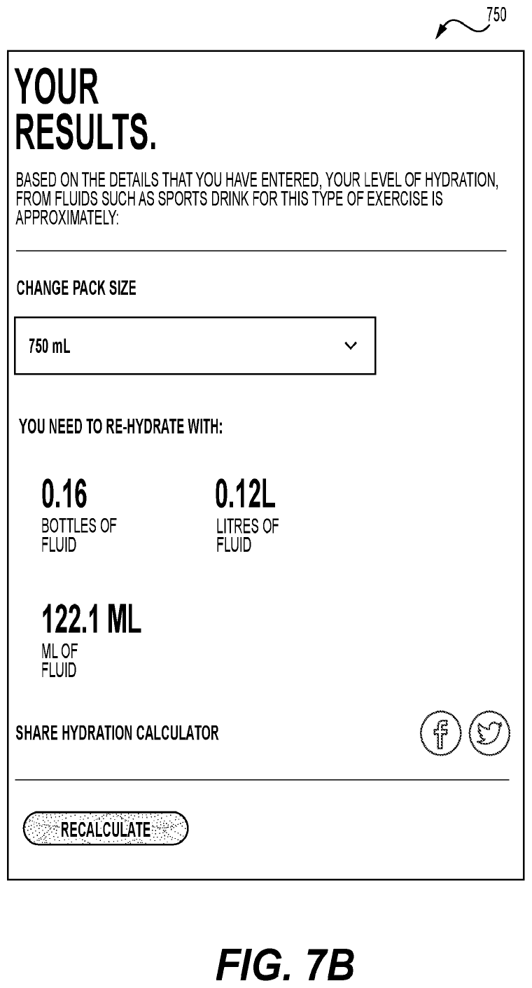

Referring now to FIGS. 7A and 7B, additional example interfaces 700 and 750 for providing activity data to generate a personalized beverage recipe are shown, according to some embodiments. Like interface 600, interfaces 700 and 750 may be examples of web interfaces (i.e., an online “hydration calculator”) accessible by users to calculate a volume of fluid and/or amount of electrolytes to be consumed to replenish those lost in sweat. Turning first to FIG. 7A, interface 700 includes fields where a user may define a type of activity (e.g., “Rugby”), a duration of the activity (e.g., 85 minutes), an ambient or outside air temperature (e.g., 14° C.), an activity intensity (e.g., “light”), a user gender (e.g., “male”), a user weight (e.g., 85 kg), and a user height (e.g., 184 cm). Once populated, the user may select a “Calculate Your Results” icon to predict a volume of fluid and/or an amount of electrolytes lost due to sweat and, subsequently, a volume of fluid and amount of electrolyte additive for a personalized beverage mixture.

In some embodiments, selecting the “Calculate Your Results” icon can cause interface 750, shown in FIG. 7B, to be presented. In some such embodiments, interface 750 is presented as a pop up or overlay to interface 700. Interface 750 may present the results of the calculations performed using the user's activity and user data. In this example, interface 750 indicates that the user should consume 122.1 mL or 0.12 L of fluid. In some embodiments, interface 750 may also present a total number of bottles (e.g., of a specific volume, such as 750 mL), cups, or other predefined measurement that the user should consume. In this example, the user has selected a bottle size of 750 mL, which is used to determine that the user should consume 0.16 bottles of fluid.

Referring now to FIG. 8, an example of a sensor for detecting a level of electrolytes lost by a user is shown, according to some embodiments. In particular, the example sensor shown may be one of the chemical sweat sensors described in U.S. Provisional Patent Application No. 63/138,964 or U.S. Provisional Patent Application No. 63/276,860, as referred to above. Accordingly, the example sensor may be worn on the skin of a user to detect a level of electrolytes in the user's sweat. The electrolyte level may be displayed via one or more bars (e.g., shown between “Low” and “High” markings), which may be colored or filled in (e.g., may change color) based on the concentration of electrolytes in the sweat.

For example, each “bar” of the sensor may correspond to an electrolyte concentration, where one bar indicates a concentration of 5-20 mmol, two bars indicate a concentration of 25-40 mmol, three bars indicate a concentration of 45-60 mmol, and four bars indicate a concentration of >65 mmol. In some embodiments, as discussed above, the electrolyte concentration indicated by the sensor may be manually identified and entered into a user interface by a user. However, in other embodiments, a camera of a user device may be used to read or scan the sensor. For example, the user device may analyze a captured image of the sensor using any appropriate image processing techniques to identify the number of bars that are colored or filled in, thereby identifying the electrolyte concentration.

Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions, and arrangement of the exemplary embodiments without departing from the scope of the present disclosure.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

1. A method for dispensing a personalized beverage mixture, the method comprising:
receiving a predicted volume of fluid lost by a user through sweat based on activity data indicating a type of activity performed and one or more parameters for the activity;
receiving a measurement of an amount of electrolytes lost by the user through sweat;
generating a recipe for the personalized beverage mixture based on the predicted volume of fluid and the measured amount of electrolytes lost by the user, the recipe comprising a second volume of fluid to replenish at least a portion of the predicted volume of fluid lost by the user and an amount of electrolyte additive to replenish at least a portion of the measured amount of electrolytes lost by the user; and
dispensing the personalized beverage mixture according to the recipe.

2. The method of claim 1, wherein the one or more activity parameters include at least one of a duration of the activity, an activity intensity, or ambient environmental conditions.

3. The method of claim 1, wherein the activity data is provided as a user input to a user device operated by the user or is automatically collected by the user device as the user performs the activity.

4. The method of claim 1, further comprising receiving physical parameters for the user including at least one of a height or a weight of the user, wherein the amount of electrolytes lost by the user is determined based further on the height or the weight of the user.

5. The method of claim 1, wherein the measurement of the amount of electrolytes lost by the user through is collected by a sensor positioned on the skin of the user.

6. The method of claim 5, wherein the sensor is a chemical electrolyte sensor configured to display a visual indicator of the amount of electrolytes lost.

7. The method of claim 5, wherein the sensor an electronic electrolyte sensor configured to wirelessly transmit or visually display an indication of the amount of electrolytes lost.

8. The method of claim 1, further comprising displaying, via a user interface, an indication of the volume of fluid for the personalized beverage mixture.

9. The method of claim 8, further comprising displaying, via the user interface, an indication of a dehydration risk for the user.

10. The method of claim 1, further comprising receiving a user input indicating at least one of a flavor or a sweetener to add to the fluid, wherein the recipe for the personalized beverage mixture further includes an amount of the flavor or the sweetener.

11. A method for dispensing a personalized beverage mixture, the method comprising:
receiving a predicted volume of fluid and a predicted amount of electrolytes lost by a user through sweat based on activity data indicating a type of activity performed and one or more parameters for the activity;
generating a recipe for the personalized beverage mixture based on the predicted volume of fluid and the predicted amount of electrolytes lost by the user, the recipe comprising a second volume of fluid to replenish at least a portion of the predicted volume of fluid lost by the user and an amount of electrolyte additive to replenish at least a portion of the predicted amount of electrolytes lost by the user; and
dispensing the personalized beverage mixture according to the recipe.

12. The method of claim 11, wherein the one or more activity parameters include at least one of a duration of the activity, an activity intensity, or ambient environmental conditions.

13. The method of claim 11, wherein the activity data is provided as a user input to a user device operated by the user or is automatically collected by the user device as the user performs the activity.

14. The method of claim 11, wherein the activity data further comprises a measurement of electrolyte loss for the user based on data received from a wearable device worn by the user.

15. The method of claim 14, wherein the wearable device is a sweat sensor positioned on the skin of the user.

16. The method of claim 14, wherein a face of the wearable device is scanned using a camera of a user device operated by the user to determine the measurement of electrolyte loss.

17. The method of claim 11, further comprising displaying, via a user interface, an indication of the volume of fluid for the personalized beverage mixture.

18. The method of claim 17, further comprising displaying, via the user interface, an indication of a dehydration risk for the user.

19. The method of claim 11, further comprising receiving physical parameters for the user including at least one of a height or a weight of the user, wherein the amount of electrolytes lost by the user is determined based further on the height or the weight of the user.

20. Computer readable media having instructions stored thereon that, when executed by one or more processors, cause the one or more processors to perform operations comprising:
receiving activity data indicating a type of activity performed by a user and one or more parameters for the activity;
predicting a first volume of fluid lost by the user through sweat during the activity;
receiving, from a sensor positioned on the skin of the user, a measurement of an amount of electrolytes lost by the user through sweat;
generating a recipe for a personalized beverage mixture based on the first volume of fluid and the measured amount of electrolytes lost by the user, the recipe comprising a second volume of fluid to replenish at least a portion of the first volume of fluid lost by the user and an amount of electrolyte additive to replenish at least a portion of the measured amount of electrolytes lost by the user; and
transmitting a control signal to a drink dispensing device to cause the drink dispensing device to dispense the personalized beverage mixture according to the recipe.
